(19) United States
(12) Patent Application Publication
(10) Pub. No.: US 2015/0148276 A1
(43) Pub. Date: May 28, 2015

(54) SANITIZER SHEETS FOR REMOVING PARTICLES FROM SKIN

(71) Applicant: Lawrence Castaldi, III, Lighthouse Point, FL (US)

(72) Inventor: Lawrence Castaldi, III, Lighthouse Point, FL (US)

(21) Appl. No.: 14/088,391

(22) Filed: Nov. 23, 2013

(51) Int. Cl. A61K 8/02; A61Q 19/10

(52) U.S. Cl. CPC A61K 8/0208; A61Q 19/10
USPC 510/138; 526/72; 526/340.2

(57) ABSTRACT

A waterless hand cleaner that pulls dirt and debris particles out of the crevices and pores of skin, thus removing harmful contaminants and bacteria along with it. The hand cleaner is a flexible sticky hand cleaner sheet with a sticky adhesive that secures and removes dirt and exfoliates loose and/or dead skin from the palms and fingers of the user's hand. The hand cleaner sheets can be dispensed from a roll, or dispensed as individual sheets.

### BACKGROUND

1. Technical Field

This invention relates in general to health. In particular, it relates to a method of avoiding unnecessary contamination and/or infections by using a flexible waterless adhesive sheet for extracting detritus and dirt particles from skin surfaces, including hands.

2. Background of the Invention

Personal hygiene has become more important as infectious agents have become more difficult to contain and suppress. This is true both for individuals in public setting, such as restaurants and mall restrooms, as well as institutional environments such as hospitals, schools, and commercial food producing facilities. In public facilities, individuals by necessity have to come in contact with items that have previously had contact with many others. As the number of individuals handling a particular item increases, the risk of infectious agents being left on that item also increases. It would be desirable to have a method of helping to protect individuals such that the risk of infection from contact with potentially infectious surfaces is reduced. It would also be desirable to have a device to protect the individual from potential contamination, and which could be easily and conveniently used by an individual as a personal item carried with them or as a product provided by an institution at the point of use

Some individuals, when using public facilities, attempt to avoid infectious agents by using disposable items such as napkins or paper towels. However, these are not always readily available for use. Further, common items such as paper towels and napkins typically do not have any antibacterial properties. Further, common cloth towels, paper towels and napkins actually force contaminants deeper into skin pores leaving germs and viral agents in a warm, moist environment perfect for breeding contagions. It would be desirable to have a device that can be used to protect an individual from potential contamination, and which could be easily and conveniently be used by an individual to eliminate infectious agents.

In addition to contaminants and infectious agents that I've always been in the environment, over time, a variety of new and deadly diseases have developed. Some of these diseases developed as the result of normal evolutionary processes. Others have developed mutations brought about as a result of exposure to a variety of antiviral and antibacterial medications that have resulted in the development of infectious agents that are either highly or completely resistant to known treatments.

For example, one such disease is brought about by MRSA (Methicillin Resistant Staphylococcus Aureus). MRSA is a bacterium that can cause infections throughout the body. It is highly resistant to many commonly used antibiotics, and is therefore more difficult to treat than most strains of staphylococcus aureus (e.g. staph). Regrettably, MRSA is only one example of many dangerous infectious agents that now represent a significant health risk to the public.

Another, even more dangerous disease is Clostridium Difficile (“C. diff”). C. diff is a species of bacteria of the genus Clostridium. Clostridia are anaerobic, spore-forming rods (bacillus). It is the most serious cause of antibiotic-associated diarrhea and can lead to other problems such as pseudomembranous colitis, which is a severe infection of the colon that results from eradication of the normal gut flora by antibiotics. When this happens, the C. diff bacteria, which naturally reside in the body, become overgrown. The overgrowth results in the release of bacterium toxins that can cause bloating, constipation, and diarrhea with abdominal pain, which may become severe.

C. diff, like MRSA, is highly infectious and in some situations, such as hospitals or nursing homes, the likelihood of passing infections from one person to another is high.

Unfortunately, these represent only two significant health problems that confront the public on a daily basis. Other infectious agents include, but are not limited to, bacteria such as E. coli, Pseudomonas Aeruginosa, Staphylococcus Typhimurium, Salmonella, Mycobacterium Tuberculosis, Yersinia Enterocolitica, and Camplyobacter jejuni. In addition to bacterial infections, there are also a variety of viral infections, including Polioviruses, Adenoviruses, Rotaviruses, Hepatitis Viruses, Influenza viruses and HIV. As can be seen, there is no shortage of dangerous infectious agents which individuals are exposed to in environments such as hospitals and nursing homes.

The pharmaceutical industry has developed a variety of pharmaceuticals that can help individuals suffering from conditions brought about by the wide variety of currently active infectious materials and contaminants. However, pharmaceuticals are needed after the fact. The after the fact treatment of infections further taxes already overloaded medical resources in their production and administration. It would be better if initial infections or contaminants were avoided, thereby saving resources and human suffering as well as potentially human life. It would be much better if initial infections or contamination can be avoided rather than treated after the fact. It would be desirable to have a method of improving the ability of individuals to avoid infection or contamination through the use of more effective personal hygiene devices.

The prior art has focused on two primary areas. The first area being the prevention of infection. This is primarily an accomplished through the use of antibacterial soaps, and protective devices such as latex gloves, etc. The second area being the use of pharmaceutical solutions to infections once they have occurred. One cause of infection that has not been addressed by the prior art is the problem associated with very small particles of dirt or detritus, typically not visible to the naked eye, which get caught in the pores or crevices of the skin. Even when individuals properly wash hands with soap, some of these particles, which are often contaminated, will remain lodged on the skin's surface. It would be desirable to remove these particles such that an individual would not be exposed to any infectious materials or contaminants that the particles carry.

While the prior art has provided basic solutions related to preventing infections through proper cleanliness, and curing infections after contamination, it has failed to provide an improved method of avoiding infections and/or contamination by eliminating small particles of detritus from the skin that would normally not be removed by conventional cleanliness procedures.

### SUMMARY OF THE INVENTION

This invention is a flexible waterless hand cleaner made specifically to pull small dirt and debris particles out of the crevices and pores of skin, thus removing harmful contaminants and bacteria along with it. The hand cleaner is a flexible sticky hand cleaner sheet with a sticky adhesive that would lock in dirt as well as exfoliate loose and/or dead skin from the palms and fingers of the user's hand as well as other skin surfaces. An individual would simply grasp the sticky hand cleaner sheet by the hand, make a fist, and then remove and discard the sheet in a trash receptacle.

### BRIEF DESCRIPTION OF THE DRAWINGS

FIG. 1 is a front view of a preferred embodiment of the flexible waterless hand cleaner sheet configured as a single sheet.

FIG. 2 is a side view of a preferred embodiment of the flexible waterless hand cleaner sheet showing multiple hand cleaner sheets configured as a pad of sheets.

FIG. 3 is a side view of a preferred embodiment of multiple flexible waterless hand cleaner sheets configured as a roll.

FIG. 4 is a front view of a preferred embodiment of a flexible waterless hand cleaner that as an optional outline of a hand on the adhesive.

### DETAILED DESCRIPTION OF THE PREFERRED EMBODIMENT

Prior to a discussion of the figures, an overview of the invention will be presented. The invention is a hand cleaner that uses a flexible sticky hand cleaner sheet with a sticky adhesive that would lock in dirt as well as exfoliate loose and/or dead skin from the palms and fingers of the user's hand. While any suitable adhesive can be used, the preferred adhesive for this product is a PSA Adhesive, (i.e, a Pressure Sensitive Adhesive) which are commonly used in products such as duct tape and adhering body side molding to automobiles. Typically, PSAs are an elastomer that is compounded with a tackifier, such as a rosin ester. Elastomers can be butyl rubber, natural rubber, silicone rubbers, acrylics (including bio-based acrylates), nitriles, ethylene-vinyl acetate (EVA), styrene copolymer adhesives, and vinyl ethers.

In the preferred embodiment, the PSA is a bio-based acrylate or natural rubber or other suitable combination of ingredients in the PSA that is safe for its intended purposes, for example, multiple daily use by a physician. This is standard practice for any product entering the medical industry. However, this product has many other potential uses by the general public, such as removing insulation contamination from skin, so safety to the user and need for different strength adhesives would likely alter the appropriate formula; always with the safety of the users in mind.

In addition to removing general types of debris, the invention also provides a valuable safety feature for workers in that it is especially effective at pulling particles of asbestos and fiberglass from the workman's skin.

The PSA could be applied to the base (or binding) element with several methods such as common tapes are made and with common manufacturing methods. One such method is spraying the adhesive on such that it is aerated in the process, giving it more coverage and body. Aerating the PSA would help the process of the adhesive to push into tiny crevices and pores in the skin to access debris.

In the preferred embodiment, the hand cleaner sheet is fabricated from a wide tape made with a sticky adhesive that would adhere to dirt as well as exfoliate loose skin from the palms and fingers of the user's hand. The surface area of the tape should preferably be slightly larger than an adult male's hand with fingers spread slightly apart. A suitable size would be approximately 8 inches by 8 inches, but the size can vary as a design choice. Those skilled in the art will recognize that while any suitable material can be used, the hand cleaner sheet would provide an additional advantage if fabricated from an antibacterial material that further protects the individual from contamination.

The substrate of the hand cleaner sheet could be made out of a variety of materials. However, it should be thin and malleable enough to distribute the adhesive for the necessary task. It should have a high tensile strength so it does not rip when peeled from the hand For example, a commercially available product, such as Tyvek™, or a variant thereof would be ideal for it's thinness, wrinkle-ability and resistance to tearing. Tyvek is used for Express Mail envelopes by the US Postal Service for its tensile strength and light weight. Tyvek will work well as a substrate with a wide variety of PSAs and is safe.

In addition to the capture of undesirable particles, the hand cleaner sheets can also incorporate antibacterial additives that will further enhance the product by killing bacteria and germs that may be on the hand. Any suitable antibacterial agent can be used as long as it is suitable for use with the PSA.

An alternative preferred embodiment envisions individual hand cleaner sheets. In this embodiment, each sheet would have a peel-off protective cover that protects the sticky surface prior to use, and can be removed when an individual needs to use the hand cleaner sheet.

In the preferred embodiment, the sheets would be porous on the side with the adhesive, and non-porous on the opposing side so they could peel off easily. Likewise, the sheets would preferably have adhesive only on the porous side since this would facilitate fabricating the sheets in the form of a roll so that they can be easily dispensed. Likewise, when the hand cleaner sheets are fabricated as individual sheets rather than portions of a roll, an additional cover sheet would be used to facilitate storage and/or stacking in a dispenser.

Those skilled in the art will recognize that while the waterless hand cleaner sheets provide an additional function not found in the prior art, it is not intended to replace pre-existing methods, such as antibacterial soap, etc. Rather, it is intended as an additional method of protection that will enhance and improve the benefits of already existing products. This is especially true in situations where infections are especially to be avoided, for example, hospitals, clinics, nursing homes, etc.

The waterless aspect of the invention is important because it helps to prevent any and verdant transfer of bacteria or other contaminants. Bacteria requires water to grow and spread. For this reason, the hand cleaner sheets have been designed as waterless devices to inhibit any accidental bacterial growth. That is the problem with prior art. When health care workers wash their hands in a sink, their hands are still wet when they touch the water faucet to turn it off. This means they are leaving some germs behind in an ideal setting for bacteria to grow while picking up the germs from everyone who has come in contact with that sink previously. After a doctor or a food service worker uses a rest room and washes their hands they not only touch the dirty faucet that are often covered with wet bacteria and germs, but they also touch the door handle on the way out that is also often covered with wet bacteria and germs.

These door handles also harbor the germs of every human that has passed through that rest room including those who did not wash their hands at all. The germs are picked up from stall handles and toilet flush handles and then transferred to door exit handles where they are picked up by the damp hands of the health care worker after they washed their hands as they leave the lavatory. This bacteria is transferred and activated by the exchange of warm moisture left by every hand that has touched that door handle. Bacteria costs us billions of dollars per year because it is not just in doctor and health care offices, but rather it is endemic to every restaurant, public restroom, and other public facilities.

Dirt particles transfer easily and provide a nurturing environment for bacteria and germs. That is why health care institutions such as The Mayo Clinic recommend scrubbing hands under water for at least 20 seconds. Germs are removed from the skin because the dirt carrying the germs is removed from the skin and it is more effective in preventing the spread of disease than antibacterial agents. In fact, anti-bacterial products are being credited for producing more dangerous and resistant strains of bacteria. However, water alone will not remove all of dirt. Even after a thorough washing, tiny particles of dirt and detritus may remain lodged in the pores and/or crevices of the skin's surface. While waste particles may be difficult to see, they can carry numerous infectious bacterial agents. Applicant's invention is designed to extract these particles even though they may not be readily visible to the eye.

Delivery systems for the waterless hand cleaner can vary. Preferably, delivery systems would be hands-off systems where the user does not have to touch anything but the waterless hand cleaner itself, thus limiting secondary transmission of any contaminants. One such delivery system would be a pad of these sheets, similar to an oversized commercially available sticky note pad. The pad could be laid on a flat surface such as a countertop, or mounted in a wall mounted dispenser. This way, a person could simply walk up to the dispenser, place a hand on the pad and without the use of another hand, peel the top sticky sheet away from the pad by pulling the hand away from the pad. This would be especially advantageous in situations where running water is not readily available and where dampness contributes to the problem. The invention provides a real advantage by being able to peel away dirt and bacteria instead of sharing it with the next user of a sink or door handle. Instead, it would be sealed it away in the adhesive.

Another delivery system would be as a roll similar to a roll of paper towels that for us separate sheets. A person could simply place the bottom of their palm at the base of a sheet and roll their hand downwards until the sheet released, and the next sheet would be ready for the other hand.

Preferably, the sheets will have an optional outline of a hand showing the user where to place their palm and fingers so that the fingers were slightly spread. This way when they made a first the extra space between the fingers would scrunch the pad and adhesive up between the fingers to clean the sides of the fingers too.

Typically, the gathering and spreading of germs with the hands is done primarily with the pads of the fingers and palms, and this is exactly where the sticky hand cleaning sheets would be most effective, so the solution to the problem is concentrated exactly where the problem exists. During the fabrication process, the sticky surface of the hand cleaner sheet may be applied using a spray adhesive to one side of the sheets. Preferably, the adhesive should be sprayed on such that an even thick layer of adhesive is a deposited on the substrate of the hand cleaner sheet. The PSA can also be applied with any manufacturing machines and processes associated with applying these types of adhesives to products such as adhesive tapes and “peel and stick” edge bandings.

In use, an individual would grasp a hand cleaner sheet and make a tightly closed first to force the gum on the sheet into the pores and crevices of the hand. The hand cleaner sheet 1 is then peeled off along with any bacteria carrying debris on any surface of the body and for other skin contaminants such as fiberglass insulation contamination, jellyfish stings or asbestos contamination on skin surfaces that washing hands alone missed.

Having discussed the invention in general, we turn now to a detailed discussion of the drawings.

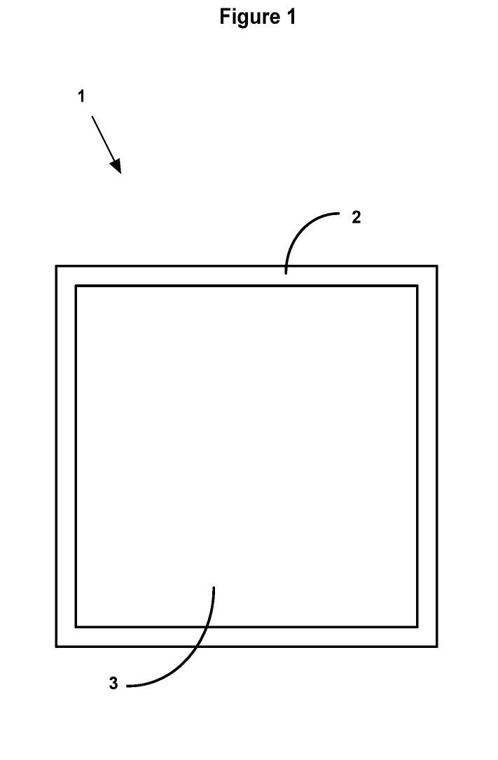

FIG. 1 is a front view of a preferred embodiment of the flexible waterless hand cleaner sheet 1 configured as a single sheet. This figure illustrates the non-porous substrate 2 and the adhesive layer 3. The non-porous layer 2 is sufficiently flexible to allow the flexible waterless hand cleaner 1 to be comfortable clenched in the hand of the user. Likewise, the adhesive layer 3 should have sufficient thickness to be able to contact the surface of the user's hand when clenched. In addition, users should extend their fingers when grasping the waterless hand cleaner sheet 1 such that the adhesive layer 3 will also contact the sides of the fingers to increase the surface area of the hand that is cleaned. Once the user finishes the cleansing process, the waterless hand cleaner sheet 1 is discarded.

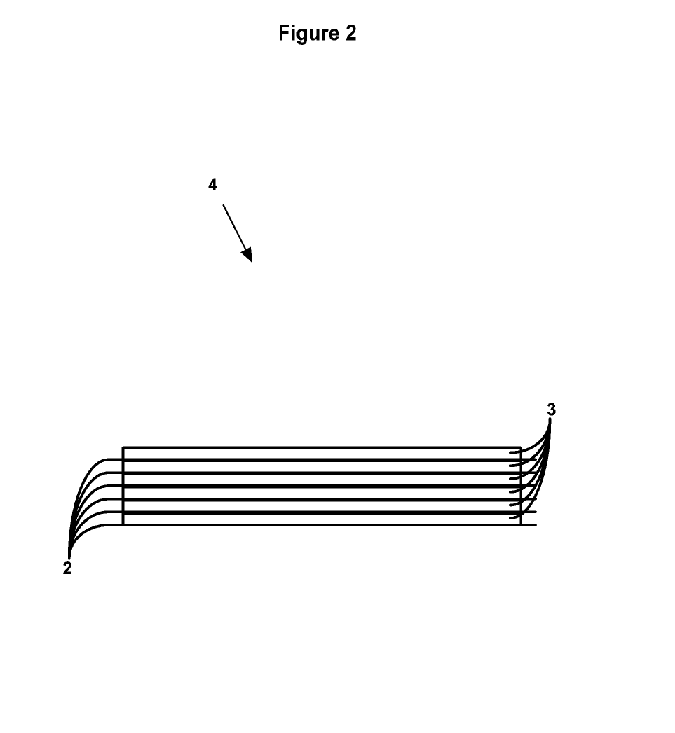

FIG. 2 is a side view of a preferred embodiment of the waterless hand cleaner sheet 1 showing multiple hand cleaner sheets 1 configured as a pad of sheets. In this configuration, the hand cleaner sheets 1 can be placed on a flat surface, such as a countertop, for easy access.

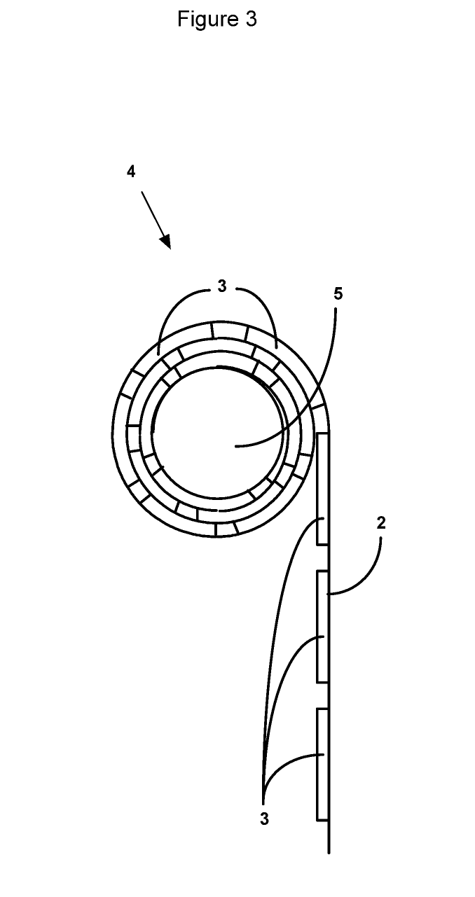

FIG. 3 is a side view of a preferred embodiment of multiple flexible waterless hand cleaner sheets 1 configured as a roll 4. In this configuration, each hand cleaner sheet 1 would be positioned on a continuous roll 4 such that individual sheets 1 can be taken off the roll 4 in the same manner as a sheet from a conventional paper towel. The figure also illustrates the spindle 5 used to hold the roll 4.

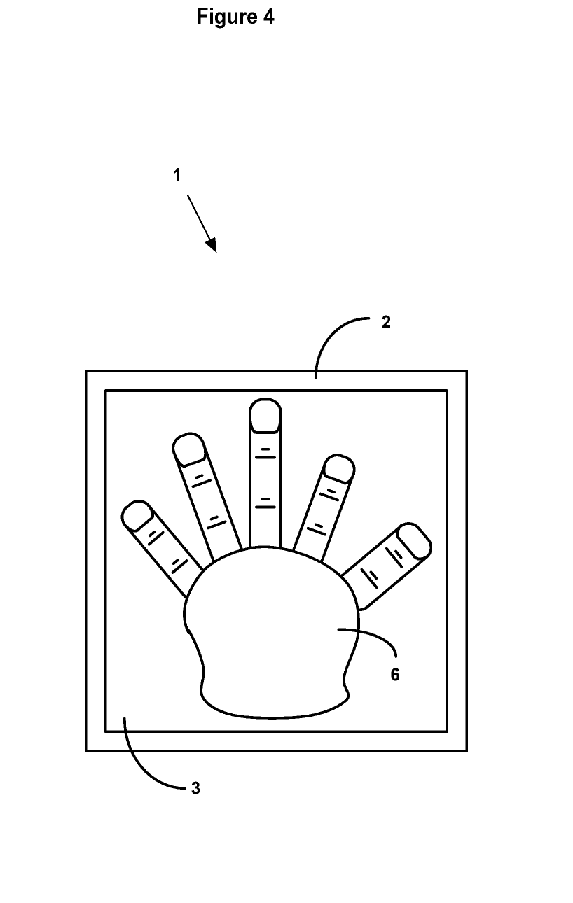

FIG. 4 is a front view of an alternative preferred embodiment of a flexible waterless hand cleaner sheet 1 that as an optional outline of a hand 6 on the adhesive layer 2. The outline serves the function of reminding users to spread their fingers when grasping the hand cleaner sheet 1. Those skilled in the art will recognize that the outline can vary based on the part of the body that the hand clean sheet 1 is in contact with. For example, when used on legs and arms it could be an elongated oval, etc.

In the preferred embodiment, the adhesive layer 3 has a gummy feel and texture that aids when it adheres to dirt or detritus on the user's hand. The gummy texture also aids in contacting the maximum amount of skin surface when the hand cleaner sheet 1 is clenched in the user's hand.

Those skilled in the art will recognize that while the discussion of the invention has centered on cleaning and individual's hands, the invention can be used on any surface of the body.

While specific embodiments have been discussed to illustrate the invention, it will be understood by those skilled in the art that variations in the embodiments can be made without departing from the spirit of the invention. The size of the hand cleaner sheets can vary, the materials used to fabricate the hand cleaner sheets can vary, the method of dispensing the sheets can vary, etc. Therefore, the invention shall be limited solely to the scope of the claims.

I claim:

1. A hand cleaner sheet, comprising:
a flexible layer of adhesive material having an adhesive that has a high level of stickiness, and a gummy texture, such that the adhesive will secure any detritus and/or dirt when the adhesive layer of adhesive material is clenched by the user.

2. A device, as in claim 1, further comprising:
a substrate that provides mechanical support for the adhesive layer.

3. A device, as in claim 2, wherein:
the substrate is non-porous.

4. A device, as in claim 3, wherein:
at least one edge of the substrate extends past the edge of the layer of flexible adhesive material.

5. A device, as in claim 2, wherein:
the hand cleaner sheet is a single sheet.

6. A device, as in claim 2, wherein:
a plurality of hand cleaner sheets are attached to a pad.

7. A device, as in claim 2, wherein:
a plurality of hand cleaner sheets are attached to one another on a roll dispenser.

8. A device, as in claim 2, further comprising:
an indicia on the flexible layer of adhesive material that indicates the proper location of the user's fingers when grasping the flexible layer of adhesive material.

9. A device, as in claim 2, wherein:
the layer of adhesive material is deposited on the substrate by spraying.
